SEQUENCE LISTING (18 sequences)

SEQ ID NO: 1 - DNA, 5807 bp, Artificial Sequence
  Description of Artificial Sequence Vector Construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggcccggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcagcccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactctagaggatcctcattgtttgcctccctgctgcggtttttcaccgaagttcatgccagtccagcgtttttgcagcagaaaagccgccgacttcggtttgcggtcgcgagtgaagatccctttcttgttaccgccaacgcgcaatatgccttgcgaggtcgcaaaatcggcgaaattccatacctgttcaccgacgacggcgctgacgcgatcaaagacgcggtgatacatatccagccatgcacactgatactcttcactccacatgtcggtgtacattgagtgcagcccggctaacgtatccacgccgtattcggtgatgataatcggctgatgcagtttctcctgccaggccagaagttctttttccagtaccttctctgccgtttccaaatcgccgctttggacataccatccgtaataacggttcaggcacagcacatcaaagagatcgctgatggtatcggtgtgagcgtcgcagaacattacattgacgcaggtgatcggacgcgtcgggtcgagtttacgcgttgcttccgccagtggcgaaatattcccgtgcacttgcggacgggtatccggttcgttggcaatactccacatcaccacgcttgggtggtttttgtcacgcgctatcagctctttaatcgcctgtaagtgcgcttgctgagtttccccgttgactgcctcttcgctgtacagttctttcggcttgttgcccgcttcgaaaccaatgcctaaagagaggttaaagccgacagcagcagtttcatcaatcaccacgatgccatgttcatctgcccagtcgagcatctcttcagcgtaagggtaatgcgaggtacggtaggagttggccccaatccagtccattaatgcgtggtcgtgcaccatcagcacgttatcgaatcctttgccacgtaagtccgcatcttcatgacgaccaaagccagtaaagtagaacggtttgtggttaatcaggaactgttggcccttcactgccactgaccggatgccgacgcgaagcgggtagatatcacactctgtctggcttttggctgtgacgcacagttcatagagataaccttcacccggttgccagaggtgcggattcaccacttgcaaagtcccgctagtgccttgtccagttgcaaccacctgttgatccgcatcacgcagttcaacgctgacatcaccattggccaccacctgccagtcaacagacgcgtggttacagtcttgcgcgacatgcgtcaccacggtgatatcgtccacccaggtgttcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctgatcaattccacagttttcgcgatccagactgaatgcccacaggccgtcgagttttttgatttcacgggttggggtttctacaggacgtaacatgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgcca

SEQ ID NO: 2 - DNA, 5765 bp, Artificial Sequence
  Description of Artificial Sequence Vector Construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggcccggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgaagcagcagaagccttgatcatcttcctttgtctcaacctgaaactcttttttttctttcattgtttgttctcttttcactgtggatgtagataattgtttttaatgaaatgaagaaatattgatttgccttttgacataattttgttaataatcttgattacaaattttagtcagtgtttgatgcatagttgcatactgcagagttgagtttggatatggccacgtcagcattatctcgttaccaaaacgtaaggtccaaactcagataatacaaacgaagcagttctttgtcactctatcatcaacatatgaaccacaccaaaaaagaacaaaatcgtagataatgatcatgcaaaaccgaccgttggatcttactttcgatttcaaaccacataaatcttagtgactgagctaaaaaactgaaattttttaaaaggcaagacctcctctgtttccatattctcaccacagaagaactcttgaggctttctcttttctctaccatggcgggatccatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgcca

SEQ ID NO: 3 - DNA, 5541 bp, Artificial Sequence
  Description of Artificial Sequence Vector Construct
gtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgggcccggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttacgtcagcattatctcgttaccaaaacgtaaggtccaaactcagataatacaaacgaagcagttctttgtcactctatcatcaacatatgaaccacaccaaaaaagaacaaaatcgtagataatgatcatgcaaaaccgaccgttggatcttactttcgatttcaaaccacataaatcttagtgactgagctaaaaaactgaaattttttaaaaggcaagacctcctctgtttccatattctcaccacagaagaactcttgaggctttctcttttctctaccatggcgggatccatgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggccaacagttcctgattaaccacaaaccgttctactttactggctttggtcgtcatgaagatgcggacttacgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaagtgcacgggaatatttcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccccagtacattaaaaacgtccgcaatgtgttattaagttgtctaagcgtcaatttgtttacaccacaatatatcctgcca

SEQ ID NO: 4 - DNA, 1602 bp, Artificial Sequence
  Description of Artificial Sequence Cassette Construct
gaattcaaatttttcgccagttctaaatatccggaaacctcttgggatgccattgcccatctatctgtaatttattgacgaaatagacgaaaaggaaggtggctcctataaagcacatcattgcgataacagaaaggccattgttgaagatacctctgctgacattggtccccaagtggaagcaccaccccatgaggagcaccgtggagtaagaagacgttcgagccacgtcgaaaaagcaagtgtgttgatgtagtatctccattgacgtaagggatgacgcacaatccaactatccatcgcaagaccattgctctatataagaaagttaatatcatttcgagtggccacgctgagctcggatggattggctccagcaaattagagtacggtccaagcacatgctgaggtaatgggcacgaaccagtatcagtcatggcactgtactggctaactgcgaggccactgaggcagtagcatgaatgatagtgatctctgttctttccaggcttatccctcaagcctccctctagtacctgagaacaaagtaggatgtattgttgcagggcaatgttatggaagagtgggccaatttggttgctctgttgtataaaatcaaatccaaacttcgcatagtccacagcagaggaagacttattcgcggtgcacccatatgaactggtgctgcaggcatcctctcctgatggccttttgcaggaatacgaggacctcaattgcttatcaacaatcgtaattaacttttgtgtgaaagcaatggcagctccctgccaaaaggagtagcaaccatcaaccaatttattagttcgtccttgaaatccgcattccactccttgacgaaaagccacccagccaatcaaactaggcaagtcaactttctctgcctcattaagcaggatcaaagcagccaatccacagaatgtatacccaccatgtgcttcagcataaggctccccagcaataccaccttcataagtttgacatcttgctatgtagtcgcctacaccttttgccagtttaaaatcaagaatattcacaaggctggcaaccgatatagcggtgtaggaagcacggacatcaatttcgccaccatcatgcattctgaaagcacctgatacatctttcatctgcagcataaaattgtacaggttgcccctattgattgatgacaatgctctttcgctccctattgtcacaagtgtatttacagcagcataagtcgtagctaggtgaggcaactgtccaggtccaccactatatccaccatctttatcctgacatcgagctaagaagtctatgatatcattctcaagatcatcatcaagtgcttcatccagcaaagcaagtggatgaaccatccagtagcatagccaagggcgattggcatctagaacatgaaaggctggtcccatatgcctcagcccaggcgtcagatactcgatatgctgatcacgccacagctctagcatgatggatttcgtgttgggcgcggccccgaagagggagcggtagatgtcgccaaccctggcctccaccttcatctgctccacctgcgtcaccgtgagcctcggtaggtcgggatcc

SEQ ID NO: 5 - DNA, 29 bp, Artificial Sequence
  Description of Artificial Sequence PCR Primer Sequence
aaaaagcttgaagcagcagaagccttgat

SEQ ID NO: 6 - DNA, 30 bp, Artificial Sequence
  Description of Artificial Sequence PCR Primer Sequence
aaaggatcccgccatggtagagaaaagaga

SEQ ID NO: 7 - DNA, 30 bp, Artificial Sequence
  Description of Artificial Sequence PCR Primer Sequence
aaaaagcttacgtcagcattatctcgttac

SEQ ID NO: 8 - DNA, 27 bp, Artificial Sequence
  Description of Artificial Sequence PCR Primer Sequence
ggatcctcattgtttgcctccctgctg

SEQ ID NO: 9 - DNA, 27 bp, Artificial Sequence
  Description of Artificial Sequence PCR Primer Sequence
gaattcatgttacgtcctgtagaaacc

SEQ ID NO: 10 - DNA, 28 bp, Artificial Sequence
  Description of Artificial Sequence PCR Primer Sequence
ggatccggcggatcccgacctaccgagg

SEQ ID NO: 11 - DNA, 35 bp, Artificial Sequence
  Description of Artificial Sequence PCR Primer Sequence
gagctcaaagagctcgtggatggattggctccagc

SEQ ID NO: 12 - DNA, 36 bp, Artificial Sequence
  Description of Artificial Sequence PCR Primer Sequence
aaagaattcaaatttttcgccagttctaaatatccg

SEQ ID NO: 13 - DNA, 36 bp, Artificial Sequence
  Description of Artificial Sequence PCR Primer Sequence
aaagagctcagcgtggccactcgaaatgatattaac

SEQ ID NO: 14 - DNA, 33 bp, Artificial Sequence
  Description of Artificial Sequence PCR Primer Sequence
ggtaccaaatttttcgccagttctaaatatccg

SEQ ID NO: 15 - DNA, 28 bp, Artificial Sequence
  Description of Artificial Sequence PCR Primer Sequence
ggtaccggcggatcccgacctaccgagg

SEQ ID NO: 16 - DNA, 23 bp, Artificial Sequence
  Description of Artificial Sequence PCR Primer Sequence
gccgacagtggtcccaaagatgg

SEQ ID NO: 17 - DNA, 24 bp, Artificial Sequence
  Description of Artificial Sequence PCR Primer Sequence
ccgcagcagggaggcaaacaatga

SEQ ID NO: 18 - DNA, 24 bp, Artificial Sequence
  Description of Artificial Sequence PCR Primer Sequence
gatgccgacgcgaagcgggtagat

(19) United States
(12) Patent Application Publication
(10) Pub. No.: US 2005/0172361 A1
(43) Pub. Date: Aug. 4, 2005

(54) Regulation of gene expression in plant cells

(76) Inventor: Yafan Huang, Kingston (CA)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/050,913

(22) Filed: Feb. 4, 2005

Related U.S. Application Data
(60) Provisional application No. 60/542,096, filed on Feb. 4, 2004.

(51) Int. Cl. A01H 1/00; C12N 15/82; C12N 5/04

(52) U.S. Cl. 800/286; 435/468; 435/419

(57) ABSTRACT

The invention relates to gene cassette constructs for expression of a nucleic acid sequence of interest. The nucleic acid sequence of interest produces an RNA transcript that is an anti-sense RNA molecule complementary to an endogenously expressed gene of the host cell. Also included are transgenic plants expressing the nucleic acid sequence of interest, and transgenic plant cells, tissues and plants having altered phenotypes resulting from the expression of a nucleic acid sequence of interest in an anti-sense orientation.

### RELATED APPLICATIONS

This application claims the benefit of U.S. Provisional Application No. 60/542,096 filed Feb. 4, 2004 which is incorporated herein by reference in its entirety.

### FIELD OF THE INVENTION

The invention relates to methods of modulating gene expression in a plant cell to produce plants with an altered phenotype

### BACKGROUND OF THE INVENTION

Transcription is the process by which the DNA genetic code is converted to a RNA for cellular distribution of the encoded product or function. Synthesis of protein-coding RNA transcript (mRNA) is mediated by RNA Polymerase II. In higher eukaryotes, all protein-coding mRNAs, with the exception of histones, have a similar transcription mechanism. RNA Polymerase II binds DNA upstream of the gene in the promoter region, synthesizes a pre-mRNA molecule through to the transcriptional termination region downstream of the gene. The terminator region is comprised of the conserved cis sequence elements that, in association with a multimeric protein complex, facilitates RNA Polymerase II dissociation from the DNA and directs pre-mRNA site-specific cleavage and polyadenylation of the newly formed 3′end.

Efficient gene expression requires a terminator sequence. The deletion of a terminator region significantly reduces efficiency. (Platt 1986 Ann Rev Biochem 55:339-372; An et al 1989 Plant Cell 1:115-122). Current methods of inhibiting gene expression include anti-sense, co-suppression and hairpin or RNAi. While not being held to a specific mechanism of action, one hypothesis of anti-sense reduction of gene expression suggests that anti-sense RNA bind and interfere with the translation of its complementary sense mRNA within plant cells. Alternatively, binding of the anti-sense RNA to a complementary endogenous RNA target and the formation of a double-stranded RNA molecule initiates a degradation mechanism targeting double-stranded RNA, thereby reducing the transcript pool available for translation. In either case, transcription of an anti-sense RNA will lead to a reduction in the expressed gene products of the target mRNA. A method of anti-sense gene inhibition in plant cells is described by Shewmaker et al. (U.S. Pat. Nos. 5,107,065 and 5,759,829). The method involves integration into the plant genome of a transcriptional active gene cassette that produces a RNA at least partially complementary to a DNA sequence endogenously transcribed by the host cell. Shewmaker et al. teaches an anti-sense gene cassette construct based on the paradigm of, 5′ to 3′, sequentially required elements consisting of a promoter, a DNA sequence being at least partially complementary to an endogenous gene of the host cell and a termination region.

### SUMMARY OF THE INVENTION

The present invention provides methods and compositions for expression of nucleic acids. In particular, the expression of anti-sense nucleic acid sequences that reduce the expression of an endogenous target gene.

In plant cells, expression of an exogenous gene has been achieved by the introduction of a gene construct consisting of a promoter, and a gene of interest. The gene construct lacks a termination region. The gene of interest can encode and express a functional gene product when the nucleic acid sequence is oriented in a sense direction and produces a translatable mRNA. Alternatively, the nucleic acid sequence, or a portion thereof, is in the anti-sense orientation thereby producing a transcript which is not translatable but has the effect of inhibiting expression or translation of a homologous, or complementary, target gene.

Accordingly, the invention provides methods of modulating the expression of an gene of interest (i.e., target gene) in a plant cell, a monocotyledon, a dicotyledon, or a gymnosperm, by introducing to the plant cell a gene construct containing a promoter operably linked to a nucleic acid sequence. The construct lacks a termination region. By termination region is meant a nucleic acid sequence that signals transcriptional termination. A termination region includes for example the opine termination region. Transcription of the nucleic acid sequence in the transformed plant cell produces a RNA transcript complementary to an endogenous RNA transcript, e.g., mRNA produced by a gene expressed in the cell. The complementary RNA interacts the endogenous RNA transcript modulating the expression of gene of interest in the transformed plant cell.

By modulating expression is meant an increase or decrease in expression of the gene compared to the expression of the gene in a cell that has not been contacted with the gene construct, e.g., a non-transformed or wild type cell. Expression is determined at the RNA level using any method known in the art. For example, Northern hybridization analysis using probes which specifically recognize one or more of these sequences can be used to determine gene expression. Alternatively, expression is measured using reverse-transcription-based PCR assays, e.g., using primers specific for the differentially expressed sequences. Expression is also determined at the protein level, i.e., by measuring the levels of polypeptides encoded by the gene products described herein. Such methods are well known in the art and include, e.g., immunoassays based on antibodies to proteins encoded by the genes.

The target gene is endogenous. Alternatively, the target gene is exogenous. The target gene is a gene in which modulation of expression is desired such as a enzyme, a metabolic gene, a housekeeping gene, a structural gene or an regulatory gene. For example, the gene is a farnesyltransferase, prenyl protese, methyl transferase, beta-glucuronidase (GUS), anthocyanidin reductase (BAN), ACC-synthase, actin, tubulin, WRKY transcription factor, or a MYB transcription factor.

The nucleic acid is single stranded. Alternatively the nucleic acid is double stranded. The nucleic acid is in the sense orientation or the ant-sense orientation. The nucleic acid sequence includes a sequence complementary to the entire RNA, i.e., full-length. Alternatively, nucleic acid sequence includes a sequence complementary to the a portion, i.e., fragment of the RNA. The nucleic acid is complementary to the coding region of the RNA. Alternatively, the nucleic acid in complementary to a non-coding region of the RNA. The promoter is any promoter that is capable of expressing the nucleic acid in a plant cell. The promoter is constitutive promoter, a tissue specific promoter or an inducible promoter.

Also included in the invention are the plant cell and plants produced by the methods of the invention and the seed produced by the plants which produce a plant that has reduced gene expression and or an altered phenotype. The cells and plants have reduced expression of the target gene and or an altered phenotype compared to a wild type plant. An altered, e.g., increased or decreases, phenotype includes for example altered stress resistance, pathogen resistance, herbicide resistance, altered flower color, altered transpiration rate, or increased fruit, seed, or biomass production.

The invention further includes a DNA containing a promoter nucleic acid sequence functional in a plant cell and a nucleic acid sequence complementary to a nucleic acid sequence encoding a target gene or fragment thereof, where the construct lacks a termination region. Also included in the invention is a plasmid containing the construct and a cell containing the plasmid. Optionally the plasmid contains a The invention a replication system functional in a prokaryotic host or Agrobacterium.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and from the claims.

### BRIEF DESCRIPTION OF THE DRAWING

FIG. 1 is a diagram of that portion of plasmid construct pBI121:anti:GUS:ΔTerm (SEQ ID NO:1) that lies between the right and left borders of the transformation plasmid, pBI121. Restriction sites used in the cloning scheme are indicated.

FIG. 2 is a diagram of that portion of plasmid construct pHPR:GUS (SEQ ID NO:2) that lies between the right and left borders of the transformation plasmid, pBI121. Restriction sites used in the cloning scheme are indicated.

FIG. 3 is a diagram of that portion of plasmid construct pHPRT:GUS (SEQ ID NO:3) that lies between the right and left borders of the transformation plasmid, pBI121. Restriction sites used in the cloning scheme are indicated.

FIG. 4 is a diagram of the terminatorless cassette portion of construct MuA:anti-ZmFT-B:ΔTerm (SEQ ID NO:4). Restriction sites used in the cloning scheme are indicated.

FIG. 5 are photographs showing PCR analysis of transgenic (three lines), parental and control lines for the presence or absence of the pBI121:antiGUS:ΔTerm construct (FIG. 5A) or the pHPR:GUS construct (FIG. 5B). The expected band for the pBI121:antiGUS:ΔTerm fragment is approximately 1.04 kb and the expected band for the pHPR:GUS fragment is approximately 1.29 kb. Abbreviations are P represents parental line, Col represents wild type Arabidopsis variety Columbia, “+” represents the positive PCR control and “−” represents the PCR negative control.

FIG. 6 are photographs showing the GUS staining analysis of transgenic (three lines), parental and control lines. Positive GUS activity in leaf tissue is marked by a blue colour and negative GUS activity in leaf tissue by a lack of blue staining. The PCR results for the GUS sense and antisense constructs are summarized by a + or − sign.

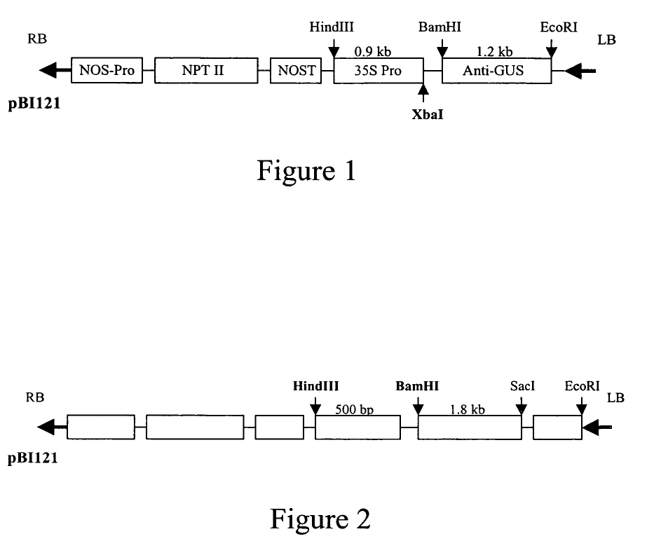

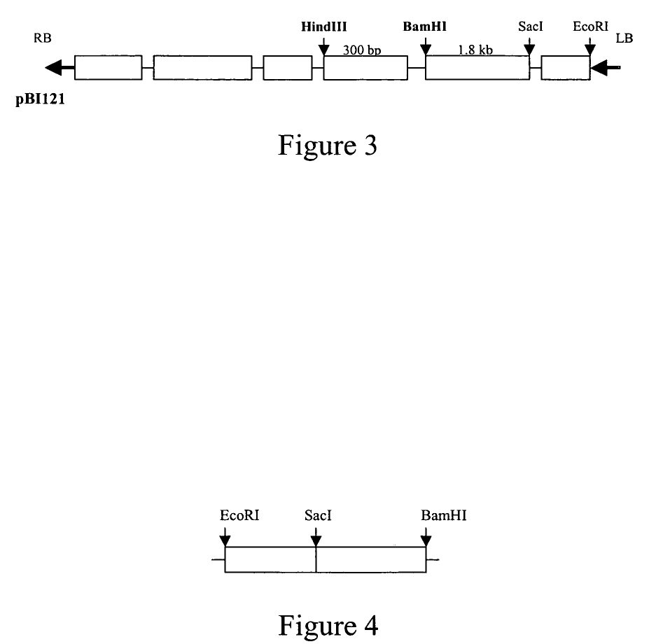

### DETAILED DESCRIPTION

The invention is based in part on the unexpected discovery that efficient reduction of endogenous gene expression is achieved using a vector containing a construct containing a in the 5′-3′ orientation a promoter that is functional in a plant cell and a DNA sequence being at least partially complementary (i.e., antisense) to an endogenous gene sequence, the construct lacking a transcriptional terminator. This result was surprising as traditional method of reducing expression of an exogenous gene in plant cell has only been achieved by the introduction of a gene construct consisting of a promoter, a gene of interest or complement thereof and a termination region.

Accordingly, the invention provide compositions and methods for modulating, e.g. increase or decrease, gene expression in a cell, e.g. a plant cell. The methods are useful in the modulation of a phenotypic property of a plant.

Compositions for Modulation Gene Expression

The compositions according to the invention include transcription constructs having a transcriptional initiation sequence, e.g. a promoter and a nucleic acid sequence. Optionally, the construct includes additional regulatory sequences such as enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell, such as 35CaMV, MuA or ubiqitin and those that direct expression of the nucleotide sequence only in certain host cells (e.g., tissue-specific regulatory sequences) such as such as a hydroxpyruvate reductase (HPR), napin, anthocyanidin reductase known as the Banylus gene (BAN) or oleosin promoter. The nucleic acid sequence and the transcriptional initiation sequence is operably linked. “Operably-linked” is intended to mean that the nucleotide sequence of interest is linked in a manner that allows for expression of the nucleotide sequence (e.g., in an in vitro transcription/translation system or in a host cell when the construct is introduced into the host cell). The transcriptional construct is oriented in the direction of transcription, e.g., 5′-3′. The nucleic acid sequence is complementary, e.g. antisense, to a sequence present on RNA, e.g., messenger RNA, endogenous to a host. The RNA is encoded by an gene of interest (i.e., target gene). The gene of interest is an exogenous gene or an endogenous gene. By an endogenous gene it is meant any gene that is present in the a parental or wild type, e.g., non-transformed cell. For example, an endogenous gene of interest includes farnesyl transferase, prenyl protease, methyl transferase, pectin methyl esterase, phosphatase, enolase, ADP-glucose-pyrophosphorylase, anthocyanidin reductase (BAN), ACC-synthase, actin, tubulin, Betagluguronidase (GUS), WRKY transcription factor, or a MYB transcription factor.

The nucleic acid sequence includes a sequence complementary to the entire endogenous RNA. Alternatively, nucleic acid sequence includes a sequence complementary to the a portion, i.e., fragment of the RNA. The nucleic acid sequence is least about 15 nucleotides, more usually at least about 20 nucleotides, preferably about 30 nucleotides, and more preferably about 50 nucleotides, and may be 100 nucleotides or more, usually being fewer than about 5000 nucleotides, more usually being fewer than 2000 nucleotides, and preferably being fewer than 1000 nucleotides. The sequence may be complementary to any sequence of the messenger RNA, that is, it may be proximal to the 5′-terminus or capping site, downstream from the capping site, between the capping site and the initiation codon and may cover all or only a portion of the non-coding region, may bridge the non-coding and coding region, be complementary to all or part of the coding region, complementary to the 3′-terminus of the coding region, or complementary to the 3′-untranslated region of the mRNA.

The particular site to which the nucleic acid sequence binds and the length of the sequence will vary depending upon the degree of modulation desired, the uniqueness of the sequence, or the stability of the nucleic acid sequence. The nucleic acid sequence is a single sequence or a repetitive sequence having two or more repetitive sequences in tandem, where the single sequence may bind to a plurality of messenger RNAs. Optionally, rather than providing for homoduplexing, heteroduplexing may be employed, where the same sequence may provide for modulation of a plurality of messenger RNAs by having regions complementary to different messenger RNAs.

The nucleic acid sequence is complementary to a unique sequence or a repeated sequence, so as to enhance the probability of binding. Thus, the nucleic acid sequence may be involved with the binding of a unique sequence, a single unit of a repetitive sequence or of a plurality of units of a repetitive sequence. The nucleic acid sequence may result in the modulation of expression of a single gene or a plurality of genes.

The transcriptional initiation region, e.g. a promoter may provide for constitutive expression or regulated expression. A large number of promoters are available which are functional in plants. These promoters are obtained from Ti- or Ri-plasmids, from plant cells, plant viruses or other hosts where the promoters are found to be functional in plants. Suitable promoters include bacterial promoter such as the octopine synthetase promoter, the nopaline synthase promoter, or the manopine synthetase promoter, viral promoters such as the cauliflower mosaic virus full length (35S) or region VI promoters or plant promoters such as the ribulose-1,6-biphosphate (RUBP) carboxylase small subunit (ssu), the β-conglycinin promoter, the phaseolin promoter, the ADH promoter, MuA promoter, ubiquitin promoter, heat-shock promoters, or tissue specific promoters such as a hydroxypyruvate reductase promoter (HPR), a napin promoter, a oleosin promoter or a Banylus gene promoter, e.g., promoters associated with fruit ripening or specific cell types such as guard cells, pollen or pistle tissues.

The transcriptional initiation region is a naturally-occurring region, a RNA polymerase binding region freed of the regulatory region, or a combination of an RNA polymerase binding region from one gene and regulatory region from a different gene. The regulatory region is responsive to a physical stimulus, such as heat, with heat shock genes, light, as with the RUBP carboxylase SSU, or the like. Alternatively, the regulatory region may be sensitive to differentiation signals, such as the β-conglycinin gene, the phaseolin gene, or is responsive to metabolites. The time and level of expression of the anti-sense RNA can have a definite effect on the phenotype produced. Thus the promoters chosen will determine the effect of the anti-sense RNA.

The various nucleic acids are joined by linkers, adapters, or directly where convenient restriction sites are available. The DNA sequences, particularly bound to a replication system, may be joined stepwise, where markers present on the replication system may be employed for selection.

Another aspect of the invention pertains to vectors, containing a transcription constructs according to the invention. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication). Some vectors have more than one origin of replication to permit replication in multiple host cells i.e. E. coli and Agrobacterium. Other vectors are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors or plant transformation vectors, binary or otherwise, which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed.

The recombinant vectors of the invention can be designed for expression of transcription constructs in prokaryotic or eukaryotic cells. For example, the constructs can be expressed in bacterial cells such as Escherichia coli, insect cells (using baculovirus expression vectors) yeast cells, plant cells or mammalian cells. Suitable host cells are discussed further in Goeddel, GENE EXPRESSION TECHNOLOGY: METHODS IN ENZYMOLOGY 185, Academic Press, San Diego, Calif. (1990).

The constructs of the invention are introduced into the host cell in a variety of ways. Of particular interest is the use of A. tumefaciens, with protoplasts, injured leaves, or other explant tissues. Other techniques which may find use include electroporation with protoplasts, liposome fusion, microinjection, particle bombardment or non-particle transformation i.e. aerosol beam injection, or the like. The particular method for transforming the plant cells is not critical to this invention.

Methods of Modulating Gene Expression

Expression of a target gene is modulated in a plant cell by inducing to a plant cell a transcriptional construct according to the invention to obtain a transformed cell. The gene is an endogenous gene or an exogenous gene. The gene is for example a farnesyl transferase, prenyl protease, methyl transferase, pectin methyl esterase, phosphatase, enolase, ADP-glucose-pyrophosphorylase, anthocyanidin reductase, ACC-synthase, actin, tubulin, WRKY transcription factor, or a MYB transcription factor. By modulation of expression is meant an increase or decrease of gene expression compared to a wild type cell, e.g., a cell that has not been contacted with the transcriptional construct. Upon introduction of the construct to the cell, transcription of the nucleic acid produces a RNA transcript that is complementary to an endogenous RNA transcript produced by the plant cell. The endogenous RNA encodes for the polypeptide produced by the gene of interest. The RNA complementary to the endogenous RNA transcript interacts with the endogenous transcript to modulate the translation of the endogenous transcript thereby modulating expression of the target gene.

By this method, various processes endogenous to the plant host cell are modulated, so that the production of individual proteins by a cell is reduced, multi-enzyme processes modulated, particular metabolic paths modulated or inhibited in preference to one or more other metabolic paths, production of non-proteinaceous products reduced, or cell differentiation modified.

A wide variety of modifications may be made in numerous types of plants. These modifications may include varying the fatty acid distribution of a fatty acid source, such as rapeseed, Cuphea or jojoba, delaying the ripening in fruits and vegetables, changing the organoleptic, storage, packaging, picking and/or processing properties of fruits and vegetables, delaying the flowering or senescing of cut flowers for bouquets, reducing the amount of one or more substances in the plant, such as caffeine, theophylline, nicotine or, altering flower color.

For changing the fatty acid distribution, target species could be coconut and palm trees, Cuphea species, rapeseed, or the like. The target genes of particular interest could be acetyl transacylase, acyl carrier protein, thioesterase, etc.

For varying the amount of nicotine, a target species could be tobacco. The target genes could be N-methylputrescine oxidase or putrescine N-methyl transferase.

For delaying the ripening in fruits, the target species could tomato or avocado. The target genes could be polygalacturonase or cellulase.

For varying the amount of caffeine, the target species could be coffee (Coffea arabica). The target gene could be 7-methylxanthine, 3-methyl transferase.

For varying the amount of theophylline, the species could be tea (Camellia sinensis). The target gene could be 1-methylxanthine 3-methyl transferase.

For altering flower color the targets could be petunia, roses, carnations, or chrysanthemums, etc. The target genes could be chalcone synthase, phenylalanine ammonia lyase, or dehydrokaempferol (flavone) hydroxylases, etc.

For altering lignin content, the targets could be loblolly pine, Douglas fir, poplar, etc. The target genes could be cinnamoyl-CoA:NADPH reductase or cinnamoyl alcohol dehydrogenase, etc.

In general, reducing the activity of one enzyme at a branch point in a metabolic pathway could allow alteration of the ratios of the products formed.

Transformed Plants Cells and Transgenic Plants

The invention includes protoplast, plants cells, plant tissue and plants (e.g., monocots and dicots) transformed with translational construct according to the invention. As used herein, “plant” is meant to include not only a whole plant but also a portion thereof (i.e., cells, and tissues, including for example, leaves, stems, shoots, roots, flowers, fruits and seeds).

The plant can be any plant type including, for example, species from the genera Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Zea, Avena, Hordeum, Secale, Triticum, Sorghum, Picea, Caco, and Populus.

In some aspects of the invention, the transformed plant is resistant to biotic and abiotic stresses, e.g., chilling stress, salt stress, heat stress, water stress, disease, grazing pests and wound healing. Additionally, the invention also includes a transgenic plant that is resistant to pathogens such as for example fungi, bacteria, nematodes, viruses and parasitic weeds. Alternatively, the transgenic plant is resistant to herbicides. By resistant is meant the plant grows under stress conditions (e.g., high salt, decreased water, low temperatures) or under conditions that normally inhibit, to some degree, the growth of an untransformed plant. Methodologies to determine plant growth or response to stress include for example, height measurements, weight measurements, leaf area, ability to flower, water use, transpiration rates and yield.

The invention also includes cells, tissues, including for example, leaves, stems, shoots, roots, flowers, fruits and seeds and the progeny derived from the transformed plant.

Numerous methods for introducing foreign genes into plants are known and can be used to insert a gene into a plant host, including biological and physical plant transformation protocols. See, for example, Miki et al., (1993) “Procedure for Introducing Foreign DNA into Plants”, In: Methods in Plant Molecular Biology and Biotechnology, Glick and Thompson, eds., CRC Press, Inc., Boca Raton, pages 67-88 and Andrew Bent in, Clough S J and Bent A F, 1998. Floral dipping: a simplified method for Agrobacterium-mediated transformation of Arabidopsis thaliana. The methods chosen vary with the host plant, and include chemical transfection methods such as calcium phosphate, polyethylene glycol (PEG) transformation, microorganism-mediated gene transfer such as Agrobacterium (Horsch, et al., Science, 227: 1229-31 (1985)), electroporation, protoplast transformation, micro-injection, flower dipping and particle or non-particle biolistic bombardment.

Agrobacterium-Mediated Transformation

The most widely utilized method for introducing an expression vector into plants is based on the natural transformation system of Agrobacterium. A. tumefaciens and A. rhizogenes are plant pathogenic soil bacteria which genetically transform plant cells. The Ti and Ri plasmids of A. tumefaciens and A. rhizogenes, respectfully, carry genes responsible for genetic transformation of plants. See, for example, Kado, Crit. Rev. Plant Sci., 10: 1-32 (1991). Descriptions of the Agrobacterium vector systems and methods for Agrobacterium-mediated gene transfer are provided in Gruber et al., supra; and Moloney, et al, Plant Cell Reports, 8: 238-242 (1989).

Transgenic Arabidopsis plants can be produced easily by the method of dipping flowering plants into an Agrobacterium culture, based on the method of Andrew Bent in, Clough S J and Bent A F, 1998. Floral dipping: a simplified method for Agrobacterium-mediated transformation of Arabidopsis thaliana. Wild type plants are grown until the plant has both developing flowers and open flowers. The plant are inverted for 1 minutes into a solution of Agrobacterium culture carrying the appropriate gene construct. Plants are then left horizontal in a tray and kept covered for two days to maintain humidity and then righted and bagged to continue growth and seed development. Mature seed was bulk harvested.

Direct Gene Transfer

A generally applicable method of plant transformation is microprojectile-mediated transformation, where DNA is carried on the surface of microprojectiles measuring about 1 to 4 mu.m. The expression vector is introduced into plant tissues with a biolistic device that accelerates the microprojectiles to speeds of 300 to 600 m/s which is sufficient to penetrate the plant cell walls and membranes. (Sanford, et al., Part. Sci. Technol., 5: 27-37 (1987); Sanford, Trends Biotech, 6: 299-302 (1988); Sanford, Physiol. Plant, 79: 206-209 (1990); Klein, et al., Biotechnology, 10: 286-291 (1992)).

Another method for physical delivery of DNA to plants is sonication of target cells as described in Zang, et al., BioTechnology, 9: 996-996 (1991). Alternatively, liposome or spheroplast fusions have been used to introduce expression vectors into plants. See, for example, Deshayes, et al., EMBO J., 4: 2731-2737 (1985); and Christou, et al., Proc. Nat'l. Acad. Sci. (USA), 84: 3962-3966 (1987). Direct uptake of DNA into protoplasts using CaCl₂ precipitation, polyvinyl alcohol or poly-L-ornithine have also been reported. See, for example, Hain, et al., Mol. Gen. Genet., 199: 161 (1985); and Draper, et al., Plant Cell Physiol., 23: 451-458 (1982).

Electroporation of protoplasts and whole cells and tissues has also been described. See, for example, Donn, et al., (1990) In: Abstracts of the VIIth Int;l. Congress on Plant Cell and Tissue Culture IAPTC, A2-38, page 53; D'Halluin et al., Plant Cell, 4: 1495-1505 (1992); and Spencer et al., Plant Mol. Biol., 24: 51-61 (1994).

Plants may also be transformed using the method of Held et al. (U.S. application Ser. No. 20010026941). The method utilizes an accelerated aerosol beam of droplettes which carries the desired molecules, DNA, into the target cells. The size of droplets produced by this method are reported to be sufficiently small as to transform bacterial cells of 1 to 2 microns in length.

Particle Wounding/Agrobacterium Delivery

Another useful basic transformation protocol involves a combination of wounding by particle bombardment, followed by use of Agrobacterium for DNA delivery, as described by Bidney, et al., Plant Mol. Biol., 18: 301-31 (1992). Useful plasmids for plant transformation include Bin 19. See Bevan, Nucleic Acids Research, 12: 8711-8721 (1984), and hereby incorporated by reference.

In general, the intact meristem transformation method involves imbibing seed for 24 hours in the dark, removing the cotyledons and root radical, followed by culturing of the meristem explants. Twenty-four hours later, the primary leaves are removed to expose the apical meristem. The explants are placed apical dome side up and bombarded, e.g., twice with particles, followed by co-cultivation with Agrobacterium. To start the co-cultivation for intact meristems, Agrobacterium is placed on the meristem. After about a 3-day co-cultivation period the meristems are transferred to culture medium with cefotaxime plus kanamycin for the NPTII selection.

The split meristem method involves imbibing seed, breaking of the cotyledons to produce a clean fracture at the plane of the embryonic axis, excising the root tip and then bisecting the explants longitudinally between the primordial leaves. The two halves are placed cut surface up on the medium then bombarded twice with particles, followed by co-cultivation with Agrobacterium. For split meristems, after bombardment, the meristems are placed in an Agrobacterium suspension for 30 minutes. They are then removed from the suspension onto solid culture medium for three day co-cultivation. After this period, the meristems are transferred to fresh medium with cefotaxime plus kanamycin for selection.

Transfer by Plant Breeding

Alternatively, once a single transformed plant has been obtained by the foregoing recombinant DNA method, conventional plant breeding methods can be used to transfer the gene and associated regulatory sequences via crossing and backcrossing. Such intermediate methods will comprise the further steps of: (1) sexually crossing the plant transformed with a transgene with a plant from a non-transgene containing taxon; (2) recovering reproductive material from the progeny of the cross; and (3) growing and selecting plants transformed with a transgene from the reproductive material. Where desirable or necessary, the agronomic characteristics of the susceptible taxon can be substantially preserved by expanding this method to include the further steps of repetitively: (1) backcrossing the progeny containing the transgene with non-transgene containing plants from the taxon; and (2) selecting for expression of a transgene activity (or an associated marker gene) among the progeny of the backcross, until the desired percentage of the characteristics of the susceptible taxon are present in the progeny along with the gene or genes imparting marker activity.

By the term “taxon” herein is meant a unit of botanical classification. It thus includes, genus, species, cultivars, varieties, variants and other minor taxonomic groups which lack a consistent nomenclature.

Regeneration of Transformants

The development or regeneration of plants from either single plant protoplasts or various explants is well known in the art (Weissbach and Weissbach, 1988). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene that encodes a polypeptide of interest introduced by Agrobacterium from leaf explants can be achieved by methods well known in the art such as described (Horsch et al., 1985). In this procedure, transformants are cultured in the presence of a selection agent and in a medium that induces the regeneration of shoots in the plant strain being transformed as described (Fraley et al., 1983). In particular, U.S. Pat. No. 5,349,124 (specification incorporated herein by reference) details the creation of genetically transformed lettuce cells and plants resulting therefrom which express hybrid crystal proteins conferring insecticidal activity against Lepidopteran larvae to such plants.

This procedure typically produces shoots within two to four months and those shoots are then transferred to an appropriate root-inducing medium containing the selective agent and an antibiotic to prevent bacterial growth. Shoots that rooted in the presence of the selective agent to form plantlets are then transplanted to soil or other media to allow the production of roots. These procedures vary depending upon the particular plant strain employed, such variations being well known in the art.

Preferably, the regenerated plants are self-pollinated to provide homozygous transgenic plants, or pollen obtained from the regenerated plants is crossed to seed-grown plants of agronomically important, preferably inbred lines. Conversely, pollen from plants of those important lines is used to pollinate regenerated plants. A transgenic plant of the present invention containing a desired polypeptide is cultivated using methods well known to one skilled in the art.

A preferred transgenic plant is an independent segregant and can transmit the transcription construct and its activity to its progeny. A more preferred transgenic plant is homozygous for the gene, and transmits that gene to all of its offspring on sexual mating. Seed from a transgenic plant may be grown in the field or greenhouse, and resulting sexually mature transgenic plants are self-pollinated to generate true breeding plants.

The invention will be further illustrated in the following non-limiting examples.

### EXAMPLE 1

### Vector Construction

pBI121 Anti-GUS:ΔTerm

The GUS gene of pBI121 was reoriented to the anti-sense orientation as follows. The binary vector pBI121 was digested with BamHI and EcoRI to excise the GUS-Nos-terminator fragment. The parent vector was purified by gel purification. The full-length GUS gene was PCR amplified using primers identified by SEQ ID NO:8 and SEQ ID NO:9 for insertion into the parent vector in the anti-sense orientation. Primers included the restriction sites BamHI and EcoRI to facilitate cloning. This anti-sense GUS fragment was ligated into the BamHI/EcoRI digested parent vector to yield the pBI121:Anti-GUS:ΔTerm construct (SEQ ID NO:1).

TABLE 1

BI121: Anti-GUS: ΔTerm (SEQ ID NO: 1)
Italicized sequences are the right and left
border repeats. Underlined sequence is the 35S
promoter and bolded sequence is the GUS anti-sense sequence.

gtttacccgccaatatatcctgtcaaacactgatagtttaaactgaaggcgggaaacgacaa
tctgatcatgagcggagaattaagggagtcacgttatgacccccgccgatgacgcgggacaa
gccgttttacgtttggaactgacagaaccgcaacgttgaaggagccactcagccgcgggttt
ctggagtttaatgagctaagcacatacgtcagaaaccattattgcgcgttcaaaagtcgcct
aaggtcactatcagctagcaaatatttcttgtcaaaaatgctccactgacgttccataaatt
cccctcggtatccaattagagtctcatattcactctcaatccaaataatctgcaccggatct
ggatcgtttcgcatgattgaacaagatggattgcacgcaggttctccggccgcttgggtgga
gaggctattcggctatgactgggcacaacagacaatcggctgctctgatgccgccgtgttcc
ggctgtcagcgcaggggcgcccggttctttttgtcaagaccgacctgtccggtgccctgaat
gaactgcaggacgaggcagcgcggctatcgtggctggccacgacgggcgttccttgcgcagc
tgtgctcgacgttgtcactgaagcgggaagggactggctgctattgggcgaagtgccggggc
aggatctcctgtcatctcaccttgctcctgccgagaaagtatccatcatggctgatgcaatg
cggcggctgcatacgcttgatccggctacctgcccattcgaccaccaagcgaaacatcgcat
cgagcgagcacgtactcggatggaagccggtcttgtcgatcaggatgatctggacgaagagc
atcaggggctcgcgccagccgaactgttcgccaggctcaaggcgcgcatgcccgacggcgat
gatctcgtcgtgacccatggcgatgcctgcttgccgaatatcatggtggaaaatggccgctt
ttctggattcatcgactgtggccggctgggtgtggcggaccgctatcaggacatagcgttgg
ctacccgtgatattgctgaagagcttggcggcgaatgggctgaccgcttcctcgtgctttac
ggtatcgccgctcccgattcgcagcgcatcgccttctatcgccttcttgacgagttcttctg
agcgggactctggggttcgaaatgaccgaccaagcgacgcccaacctgccatcacgagattt
cgattccaccgccgccttctatgaaaggttgggcttcggaatcgttttccgggacgccggct
ggatgatcctccagcgcggggatctcatgctggagttcttcgcccacgggatctctgcggaa
caggcggtcgaaggtgccgatatcattacgacagcaacggccgacaagcacaacgccacgat
cctgagcgacaatatgatcgggcccggcgtccacatcaacggcgtcggcggcgactgcccag
gcaagaccgagatgcaccgcgatatcttgctgcgttcggatattttcgtggagttcccgcca
cagacccggatgatccccgatcgttcaaacatttggcaataaagtttcttaagattgaatcc
tgttgccggtcttgcgatgattatcatataatttctgttgaattacgttaagcatgtaataa
ttaacatgtaatgcatgacgttatttatgagatgggtttttatgattagagtcccgcaatta
tacatttaatacgcgatagaaaacaaaatatagcgcgcaaactaggataaattatcgcgcgc
ggtgtcatctatgttactagatcgggcctcctgtcaatgctggcggcggctctggtggtggt
tctggtggcggctctgagggtggtggctctgagggtggcggttctgagggtggcggctctga
gggaggcggttccggtggtggctctggttccggtgattttgattatgaaaagatggcaaacg
ctaataagggggctatgaccgaaaatgccgatgaaaacgcgctacagtctgacgctaaaggc
aaacttgattctgtcgctactgattacggtgctgctatcgatggtttcattggtgacgtttc
cggccttgctaatggtaatggtgctactggtgattttgctggctctaattcccaaatggctc
aagtcggtgacggtgataattcacctttaatgaataatttccgtcaatatttaccttccctc
cctcaatcggttgaatgtcgcccttttgtctttggcccaatacgcaaaccgcctctccccgc
gcgttggccgattcattaatgcagctggcacgacaggtttcccgactggaaagcgggcagtg
agcgcaacgcaattaatgtgagttagctcactcattaggcaccccaggctttacactttatg
cttccggctcgtatgttgtgtggaattgtgagcggataacaatttcacacaggaaacagcta
tgaccatgattacgccaagcttgcatgcctgcagcccacagatggttagagaggcttacgca
gcaggtctcatcaagacgatctacccgagcaataatctccaggaaatcaaataccttcccaa
gaaggttaaagatgcagtcaaaagattcaggactaactgcatcaagaacacagagaaagata
tatttctcaagatcagaagtactattccagtatggacgattcaaggcttgcttcacaaacca
aggcaagtaatagagattggagtctctaaaaaggtagttcccactgaatcaaaggccatgga
gtcaaagattcaaatagaggacctaacagaactcgccgtaaagactggcgaacagttcatac
agagtctcttacgactcaatgacaagaagaaaatcttcgtcaacatggtggagcacgacaca
cttgtctactccaaaaatatcaaagatacagtctcagaagaccaaagggcaattgagacttt
tcaacaaagggtaatatccggaaacctcctcggattccattgcccagctatctgtcacttta
ttgtgaagatagtggaaaaggaaggtggctcctacaaatgccatcattgcgataaaggaaag
gccatcgttgaagatgcctctgccgacagtggtcccaaagatggacccccacccacgaggag
catcgtggaaaaagaagacgttccaaccacgtcttcaaagcaagtggattgatgtgatatct
ccactgacgtaagggatgacgcacaatcccactatccttcgcaagacccttcctctatataa
ggaagttcatttcatttggagagaacacgggggactctagaggatcctcattgtttgcctcc
ctgctgcggtttttcaccgaagttcatgccagtccagcgtttttgcagcagaaaagccgccg
acttcggtttgcggtcgcgagtgaagatccctttcttgttaccgccaacgcgcaatatgcct
tgcgaggtcgcaaaatcggcgaaattccatacctgttcaccgacgacggcgctgacgcgatc
aaagacgcggtgatacatatccagccatgcacactgatactcttcactccacatgtcggtgt
acattgagtgcagcccggctaacgtatccacgccgtattcggtgatgataatcggctgatgc
agtttctcctgccaggccagaagttctttttccagtaccttctctgccgtttccaaatcgcc
gctttggacataccatccgtaataacggttcaggcacagcacatcaaagagatcgctgatgg
tatcggtgtgagcgtcgcagaacattacattgacgcaggtgatcggacgcgtcgggtcgagt
ttacgcgttgcttccgccagtggcgaaatattcccgtgcacttgcggacgggtatccggttc
gttggcaatactccacatcaccacgcttgggtggtttttgtcacgcgctatcagctctttaa
tcgcctgtaagtgcgcttgctgagtttccccgttgactgcctcttcgctgtacagttctttc
ggcttgttgcccgcttcgaaaccaatgcctaaagagaggttaaagccgacagcagcagtttc
atcaatcaccacgatgccatgttcatctgcccagtcgagcatctcttcagcgtaagggtaat
gcgaggtacggtaggagttggccccaatccagtccattaatgcgtggtcgtgcaccatcagc
acgttatcgaatcctttgccacgtaagtccgcatcttcatgacgaccaaagccagtaaagta
gaacggtttgtggttaatcaggaactgttggcccttcactgccactgaccggatgccgacgc
gaagcgggtagatatcacactctgtctggcttttggctgtgacgcacagttcatagagataa
ccttcacccggttgccagaggtgcggattcaccacttgcaaagtcccgctagtgccttgtcc
agttgcaaccacctgttgatccgcatcacgcagttcaacgctgacatcaccattggccacca
cctgccagtcaacagacgcgtggttacagtcttgcgcgacatgcgtcaccacggtgatatcg
tccacccaggtgttcggcgtggtgtagagcattacgctgcgatggattccggcatagttaaa
gaaatcatggaagtaagactgctttttcttgccgttttcgtcggtaatcaccattcccggcg
ggatagtctgccagttcagttcgttgttcacacaaacggtgatacgtacacttttcccggca
ataacatacggcgtgacatcggcttcaaatggcgtatagccgccctgatgctccatcacttc
ctgattattgacccacactttgccgtaatgagtgaccgcatcgaaacgcagcacgatacgct
ggcctgcccaacctttcggtataaagacttcgcgctgataccagacgttgcccgcataatta
cgaatatctgcatcggcgaactgatcgttaaaactgcctggcacagcaattgcccggctttc
ttgtaacgcgctttcccaccaacgctgatcaattccacagttttcgcgatccagactgaatg
cccacaggccgtcgagttttttgatttcacgggttggggtttctacaggacgtaacatgaat
tcactggccgtcgttttacaacgtcgtgactgggaaaaccctggcgttacccaacttaatcg
ccttgcagcacatccccctttcgccagctggcgtaatagcgaagaggcccgcaccgatcgcc
cttcccaacagttgcgcagcctgaatggcgcccgctcctttcgctttcttcccttcctttct
cgccacgttcgccggctttccccgtcaagctctaaatcgggggctccctttagggttccgat
ttagtgctttacggcacctcgaccccaaaaaacttgatttgggtgatggttcacgtagtggg
ccatcgccctgatagacggtttttcgccctttgacgttggagtccacgttctttaatagtgg
actcttgttccaaactggaacaacactcaaccctatctcgggctattcttttgatttataag
ggattttgccgatttcggaaccaccatcaaacaggattttcgcctgctggggcaaaccagcg
tggaccgcttgctgcaactctctcagggccaggcggtgaagggcaatcagctgttgcccgtc
tcactggtgaaaagaaaaaccaccccagtacattaaaaacgtccgcaatgtgttattaagtt
gtctaagcgtcaatttgtttacaccacaatatatcctgcca

pHPR:GUS

The HPR promoter was PCR amplified from genomic DNA isolated from wild type Arabidopsis thaliana (ecotype Columbia) using primers which were designed based on sequence data in Accession number AC012563. The primer pair identified by SEQ ID NO:5 and SEQ ID NO:6 were used to PCR amplify the promoter and the first 2 codons of the HPR gene. The DNA fragment was cloned into pBluescript T/A vector at the EcoRV site and sequenced. The fragment was cloned into pBI121 (Clontech) at the HindIII and BamHI sites, replacing the 35S promoter of that plasmid. A truncated version of the promoter was produced using the primer pair identified by SEQ ID NO:7 and SEQ ID NO:6 and cloned as above. The resulting plasmids are referred to as pHPR:GUS (SEQ ID NO:2) and pHPRT-GUS (SEQ ID NO:3) respectively.

TABLE 2

pHPR:GUS (SEQ ID NO:2)
Italicized sequences are the right and left border repeats. Underlined sequence is
the HPR promoter and bolded sequence is the GUS sequence.
Courier font is the NOS terminator.

gtttacccgccaatatatcctgtcaaacactgatagtttaaactgaaggcgggaaacgacaatctgatcatgagcggagaattaaggga
gtcacgttatgacccccgccgatgacgcgggacaagccgttttacgtttggaactgacagaaccgcaacgttgaaggagccactcagcc
gcgggtttctggagtttaatgagctaagcacatacgtcagaaaccattattgcgcgttcaaaagtcgcctaaggtcactatcagctagc
aaatatttcttgtcaaaaatgctccactgacgttccataaattcccctcggtatccaattagagtctcatattcactctcaatccaaat
aatctgcaccggatctggatcgtttcgcatgattgaacaagatggattgcacgcaggttctccggccgcttgggtggagaggctattcg
gctatgactgggcacaacagacaatcggctgctctgatgccgccgtgttccggctgtcagcgcaggggcgcccggttctttttgtcaag
accgacctgtccggtgccctgaatgaactgcaggacgaggcagcgcggctatcgtggctggccacgacgggcgttccttgcgcagctgt
gctcgacgttgtcactgaagcgggaagggactggctgctattgggcgaagtgccggggcaggatctcctgtcatctcaccttgctcctg
ccgagaaagtatccatcatggctgatgcaatgcggcggctgcatacgcttgatccggctacctgcccattcgaccaccaagcgaaacat
cgcatcgagcgagcacgtactcggatggaagccggtcttgtcgatcaggatgatctggacgaagagcatcaggggctcgcgccagccga
actgttcgccaggctcaaggcgcgcatgcccgacggcgatgatctcgtcgtgacccatggcgatgcctgcttgccgaatatcatggtgg
aaaatggccgcttttctggattcatcgactgtggccggctgggtgtggcggaccgctatcaggacatagcgttggctacccgtgatatt
gctgaagagcttggcggcgaatgggctgaccgcttcctcgtgctttacggtatcgccgctcccgattcgcagcgcatcgccttctatcg
ccttcttgacgagttcttctgagcgggactctggggttcgaaatgaccgaccaagcgacgcccaacctgccatcacgagatttcgattc
caccgccgccttctatgaaaggttgggcttcggaatcgttttccgggacgccggctggatgatcctccagcgcggggatctcatgctgg
agttcttcgcccacgggatctctgcggaacaggcggtcgaaggtgccgatatcattacgacagcaacggccgacaagcacaacgccacg
atcctgagcgacaatatgatcgggcccggcgtccacatcaacggcgtcggcggcgactgcccaggcaagaccgagatgcaccgcgatat
cttgctgcgttcggatattttcgtggagttcccgccacagacccggatgatccccgatcgttcaaacatttggcaataaagtttcttaa
gattgaatcctgttgccggtcttgcgatgattatcatataatttctgttgaattacgttaagcatgtaataattaacatgtaatgcatg
acgttatttatgagatgggtttttatgattagagtcccgcaattatacatttaatacgcgatagaaaacaaaatatagcgcgcaaacta
ggataaattatcgcgcgcggtgtcatctatgttactagatcgggcctcctgtcaatgctggcggcggctctggtggtggttctggtggc
ggctctgagggtggtggctctgagggtggcggttctgagggtggcggctctgagggaggcggttccggtggtggctctggttccggtga
ttttgattatgaaaagatggcaaacgctaataagggggctatgaccgaaaatgccgatgaaaacgcgctacagtctgacgctaaaggca
aacttgattctgtcgctactgattacggtgctgctatcgatggtttcattggtgacgtttccggccttgctaatggtaatggtgctact
ggtgattttgctggctctaattcccaaatggctcaagtcggtgacggtgataattcacctttaatgaataatttccgtcaatatttacc
ttccctccctcaatcggttgaatgtcgcccttttgtctttggcccaatacgcaaaccgcctctccccgcgcgttggccgattcattaat
gcagctggcacgacaggtttcccgactggaaagcgggcagtgagcgcaacgcaattaatgtgagttagctcactcattaggcaccccag
gctttacactttatgcttccggctcgtatgttgtgtggaattgtgagcggataacaatttcacacaggaaacagctatgaccatgatta
cgccaagcttgaagcagcagaagccttgatcatcttcctttgtctcaacctgaaactcttttttttctttcattgtttgttctcttttc
actgtggatgtagataattgtttttaatgaaatgaagaaatattgatttgccttttgacataattttgttaataatcttgattacaaat
tttagtcagtgtttgatgcatagttgcatactgcagagttgagtttggatatggccacgtcagcattatctcgttaccaaaacgtaagg
tccaaactcagataatacaaacgaagcagttctttgtcactctatcatcaacatatgaaccacaccaaaaaagaacaaaatcgtagata
atgatcatgcaaaaccgaccgttggatcttactttcgatttcaaaccacataaatcttagtgactgagctaaaaaactgaaatttttta
aaaggcaagacctcctctgtttccatattctcaccacagaagaactcttgaggctttctcttttctctaccatggcgggatccatgtta
cgtcctgtagaaaccccaacccgtgaaatcaaaaaactcgacggcctgtgggcattcagtctggatcgcgaaaactgtggaattgatca
gcgttggtgggaaagcgcgttacaagaaagccgggcaattgctgtgccaggcagttttaacgatcagttcgccgatgcagatattcgta
attatgcgggcaacgtctggtatcagcgcgaagtctttataccgaaaggttgggcaggccagcgtatcgtgctgcgtttcgatgcggtc
actcattacggcaaagtgtgggtcaataatcaggaagtgatggagcatcagggcggctatacgccatttgaagccgatgtcacgccgta
tgttattgccgggaaaagtgtacgtatcaccgtttgtgtgaacaacgaactgaactggcagactatcccgccgggaatggtgattaccg
acgaaaacggcaagaaaaagcagtcttacttccatgatttctttaactatgccggaatccatcgcagcgtaatgctctacaccacgccg
aacacctgggtggacgatatcaccgtggtgacgcatgtcgcgcaagactgtaaccacgcgtctgttgactggcaggtggtggccaatgg
tgatgtcagcgttgaactgcgtgatgcggatcaacaggtggttgcaactggacaaggcactagcgggactttgcaagtggtgaatccgc
acctctggcaaccgggtgaaggttatctctatgaactgtgcgtcacagccaaaagccagacagagtgtgatatctacccgcttcgcgtc
ggcatccggtcagtggcagtgaagggccaacagttcctgattaaccacaaaccgttctactttactggctttggtcgtcatgaagatgc
ggacttacgtggcaaaggattcgataacgtgctgatggtgcacgaccacgcattaatggactggattggggccaactcctaccgtacct
cgcattacccttacgctgaagagatgctcgactgggcagatgaacatggcatcgtggtgattgatgaaactgctgctgtcggctttaac
ctctctttaggcattggtttcgaagcgggcaacaagccgaaagaactgtacagcgaagaggcagtcaacggggaaactcagcaagcgca
cttacaggcgattaaagagctgatagcgcgtgacaaaaaccacccaagcgtggtgatgtggagtattgccaacgaaccggatacccgtc
cgcaagtgcacgggaatatttcgccactggcggaagcaacgcgtaaactcgacccgacgcgtccgatcacctgcgtcaatgtaatgttc
tgcgacgctcacaccgataccatcagcgatctctttgatgtgctgtgcctgaaccgttattacggatggtatgtccaaagcggcgattt
ggaaacggcagagaaggtactggaaaaagaacttctggcctggcaggagaaactgcatcagccgattatcatcaccgaatacggcgtgg
atacgttagccgggctgcactcaatgtacaccgacatgtggagtgaagagtatcagtgtgcatggctggatatgtatcaccgcgtcttt
gatcgcgtcagcgccgtcgtcggtgaacaggtatggaatttcgccgattttgcgacctcgcaaggcatattgcgcgttggcggtaacaa
gaaagggatcttcactcgcgaccgcaaaccgaagtcggcggcttttctgctgcaaaaacgctggactggcatgaacttcggtgaaaaac
cgcagcagggaggcaaacaatgagagctcgaatttccccgatcgttcaaacatttggcaataaagtttcttaagattgaatcctgttgc
cggtcttgcgatgattatcatataatttctgttgaattacgttaagcatgtaataattaacatgtaatgcatgacgttatttatgagat
gggtttttatgattagagtcccgcaattatacatttaatacgcgatagaaaacaaaatatagcgcgcaaactaggataaattatcgcgc
gcggtgtcatctatgttactagatcgggaattcactggccgtcgttttacaacgtcgtgactgggaaaaccctggcgttacccaactta
atcgccttgcagcacatccccctttcgccagctggcgtaatagcgaagaggcccgcaccgatcgcccttcccaacagttgcgcagcctg
aatggcgcccgctcctttcgctttcttcccttcctttctcgccacgttcgccggctttccccgtcaagctctaaatcgggggctccctt
tagggttccgatttagtgctttacggcacctcgaccccaaaaaacttgatttgggtgatggttcacgtagtgggccatcgccctgatag
acggtttttcgccctttgacgttggagtccacgttctttaatagtggactcttgttccaaactggaacaacactcaaccctatctcggg
ctattcttttgatttataagggattttgccgatttcggaaccaccatcaaacaggattttcgcctgctggggcaaaccagcgtggaccg
cttgctgcaactctctcagggccaggcggtgaagggcaatcagctgttgcccgtctcactggtgaaaagaaaaaccaccccagtacatt
aaaaacgtccgcaatgtgttattaagttgtctaagcgtcaatttgtttacaccacaatatatcctgcca

TABLE 3

pHPRT-GUS (SEQ ID NO:3)
Italicized sequences are the right and left border repeats. Underlined sequence is
the truncated HPR (HPRT) promoter and bolded sequence is the GUS sequence.
Courier font is the NOS terminator.

gtttacccgccaatatatcctgtcaaacactgatagtttaaactgaaggcgggaaacgacaatctgatcatgagcggagaattaagggag
tcacgttatgacccccgccgatgacgcgggacaagccgttttacgtttggaactgacagaaccgcaacgttgaaggagccactcagccgc
gggtttctggagtttaatgagctaagcacatacgtcagaaaccattattgcgcgttcaaaagtcgcctaaggtcactatcagctagcaaa
tatttcttgtcaaaaatgctccactgacgttccataaattcccctcggtatccaattagagtctcatattcactctcaatccaaataatc
tgcaccggatctggatcgtttcgcatgattgaacaagatggattgcacgcaggttctccggccgcttgggtggagaggctattcggctat
gactgggcacaacagacaatcggctgctctgatgccgccgtgttccggctgtcagcgcaggggcgcccggttctttttgtcaagaccgac
ctgtccggtgccctgaatgaactgcaggacgaggcagcgcggctatcgtggctggccacgacgggcgttccttgcgcagctgtgctcgac
gttgtcactgaagcgggaagggactggctgctattgggcgaagtgccggggcaggatctcctgtcatctcaccttgctcctgccgagaaa
gtatccatcatggctgatgcaatgcggcggctgcatacgcttgatccggctacctgcccattcgaccaccaagcgaaacatcgcatcgag
cgagcacgtactcggatggaagccggtcttgtcgatcaggatgatctggacgaagagcatcaggggctcgcgccagccgaactgttcgcc
aggctcaaggcgcgcatgcccgacggcgatgatctcgtcgtgacccatggcgatgcctgcttgccgaatatcatggtggaaaatggccgc
ttttctggattcatcgactgtggccggctgggtgtggcggaccgctatcaggacatagcgttggctacccgtgatattgctgaagagctt
ggcggcgaatgggctgaccgcttcctcgtgctttacggtatcgccgctcccgattcgcagcgcatcgccttctatcgccttcttgacgag
ttcttctgagcgggactctggggttcgaaatgaccgaccaagcgacgcccaacctgccatcacgagatttcgattccaccgccgccttct
atgaaaggttgggcttcggaatcgttttccgggacgccggctggatgatcctccagcgcggggatctcatgctggagttcttcgcccacg
ggatctctgcggaacaggcggtcgaaggtgccgatatcattacgacagcaacggccgacaagcacaacgccacgatcctgagcgacaata
tgatcgggcccggcgtccacatcaacggcgtcggcggcgactgcccaggcaagaccgagatgcaccgcgatatcttgctgcgttcggata
ttttcgtggagttcccgccacagacccggatgatccccgatcgttcaaacatttggcaataaagtttcttaagattgaatcctgttgccg
gtcttgcgatgattatcatataatttctgttgaattacgttaagcatgtaataattaacatgtaatgcatgacgttatttatgagatggg
tttttatgattagagtcccgcaattatacatttaatacgcgatagaaaacaaaatatagcgcgcaaactaggataaattatcgcgcgcgg
tgtcatctatgttactagatcgggcctcctgtcaatgctggcggcggctctggtggtggttctggtggcggctctgagggtggtggctct
gagggtggcggttctgagggtggcggctctgagggaggcggttccggtggtggctctggttccggtgattttgattatgaaaagatggca
aacgctaataagggggctatgaccgaaaatgccgatgaaaacgcgctacagtctgacgctaaaggcaaacttgattctgtcgctactgat
tacggtgctgctatcgatggtttcattggtgacgtttccggccttgctaatggtaatggtgctactggtgattttgctggctctaattcc
caaatggctcaagtcggtgacggtgataattcacctttaatgaataatttccgtcaatatttaccttccctccctcaatcggttgaatgt
cgcccttttgtctttggcccaatacgcaaaccgcctctccccgcgcgttggccgattcattaatgcagctggcacgacaggtttcccgac
tggaaagcgggcagtgagcgcaacgcaattaatgtgagttagctcactcattaggcaccccaggctttacactttatgcttccggctcgt
atgttgtgtggaattgtgagcggataacaatttcacacaggaaacagctatgaccatgattacgccaagcttacgtcagcattatctcgt
taccaaaacgtaaggtccaaactcagataatacaaacgaagcagttctttgtcactctatcatcaacatatgaaccacaccaaaaaagaa
caaaatcgtagataatgatcatgcaaaaccgaccgttggatcttactttcgatttcaaaccacataaatcttagtgactgagctaaaaaa
ctgaaattttttaaaaggcaagacctcctctgtttccatattctcaccacagaagaactcttgaggctttctcttttctctaccatggcg
ggatccatgttacgtcctgtagaaaccccaacccgtgaaatcaaaaaactcgacggcctgtgggcattcagtctggatcgcgaaaactgt
ggaattgatcagcgttggtgggaaagcgcgttacaagaaagccgggcaattgctgtgccaggcagttttaacgatcagttcgccgatgca
gatattcgtaattatgcgggcaacgtctggtatcagcgcgaagtctttataccgaaaggttgggcaggccagcgtatcgtgctgcgtttc
gatgcggtcactcattacggcaaagtgtgggtcaataatcaggaagtgatggagcatcagggcggctatacgccatttgaagccgatgtc
acgccgtatgttattgccgggaaaagtgtacgtatcaccgtttgtgtgaacaacgaactgaactggcagactatcccgccgggaatggtg
attaccgacgaaaacggcaagaaaaagcagtcttacttccatgatttctttaactatgccggaatccatcgcagcgtaatgctctacacc
acgccgaacacctgggtggacgatatcaccgtggtgacgcatgtcgcgcaagactgtaaccacgcgtctgttgactggcaggtggtggcc
aatggtgatgtcagcgttgaactgcgtgatgcggatcaacaggtggttgcaactggacaaggcactagcgggactttgcaagtggtgaat
ccgcacctctggcaaccgggtgaaggttatctctatgaactgtgcgtcacagccaaaagccagacagagtgtgatatctacccgcttcgc
gtcggcatccggtcagtggcagtgaagggccaacagttcctgattaaccacaaaccgttctactttactggctttggtcgtcatgaagat
gcggacttacgtggcaaaggattcgataacgtgctgatggtgcacgaccacgcattaatggactggattggggccaactcctaccgtacc
tcgcattacccttacgctgaagagatgctcgactgggcagatgaacatggcatcgtggtgattgatgaaactgctgctgtcggctttaac
ctctctttaggcattggtttcgaagcgggcaacaagccgaaagaactgtacagcgaagaggcagtcaacggggaaactcagcaagcgcac
ttacaggcgattaaagagctgatagcgcgtgacaaaaaccacccaagcgtggtgatgtggagtattgccaacgaaccggatacccgtccg
caagtgcacgggaatatttcgccactggcggaagcaacgcgtaaactcgacccgacgcgtccgatcacctgcgtcaatgtaatgttctgc
gacgctcacaccgataccatcagcgatctctttgatgtgctgtgcctgaaccgttattacggatggtatgtccaaagcggcgatttggaa
acggcagagaaggtactggaaaaagaacttctggcctggcaggagaaactgcatcagccgattatcatcaccgaatacggcgtggatacg
ttagccgggctgcactcaatgtacaccgacatgtggagtgaagagtatcagtgtgcatggctggatatgtatcaccgcgtctttgatcgc
gtcagcgccgtcgtcggtgaacaggtatggaatttcgccgattttgcgacctcgcaaggcatattgcgcgttggcggtaacaagaaaggg
atcttcactcgcgaccgcaaaccgaagtcggcggcttttctgctgcaaaaacgctggactggcatgaacttcggtgaaaaaccgcagcag
ggaggcaaacaatgagagctcgaatttccccgatcgttcaaacatttggcaataaagtttcttaagattgaatcctgttgccggtcttgc
gatgattatcatataatttctgttgaattacgttaagcatgtaataattaacatgtaatgcatgacgttatttatgagatgggtttttat
gattagagtcccgcaattatacatttaatacgcgatagaaaacaaaatatagcgcgcaaactaggataaattatcgcgcgcggtgtcatc
tatgttactagatcgggaattcactggccgtcgttttacaacgtcgtgactgggaaaaccctggcgttacccaacttaatcgccttgcag
cacatccccctttcgccagctggcgtaatagcgaagaggcccgcaccgatcgcccttcccaacagttgcgcagcctgaatggcgcccgct
cctttcgctttcttcccttcctttctcgccacgttcgccggctttccccgtcaagctctaaatcgggggctccctttagggttccgattt
agtgctttacggcacctcgaccccaaaaaacttgatttgggtgatggttcacgtagtgggccatcgccctgatagacggtttttcgccct
ttgacgttggagtccacgttctttaatagtggactcttgttccaaactggaacaacactcaaccctatctcgggctattcttttgattta
taagggattttgccgatttcggaaccaccatcaaacaggattttcgcctgctggggcaaaccagcgtggaccgcttgctgcaactctctc
agggccaggcggtgaagggcaatcagctgttgcccgtctcactggtgaaaagaaaaaccaccccagtacattaaaaacgtccgcaatgtg
ttattaagttgtctaagcgtcaatttgtttacaccacaatatatcctgcca

MuA-anti-ZmFTBΔTerm

A terminator-less anti-sense corn farnesyl transferase β-subunit construct driven by a MuA promoter was constructed in a binary Ti vector, (MuA-anti-ZmFTBΔTerm) for introduction into corn and constructed as follows. A 1247 bp cDNA fragment encoding corn FT-B was amplified by RT-PCR from corn leaf total RNA using primers identified by SEQ ID NO:10 and SEQ ID NO:11 This BamHI-SacI fragment was then cloned into a pGEM4 vector (pGEM-anti-FTB). The corn MuA promoter was amplified by PCR using primers identified by SEQ ID NO:12 and SEQ ID NO:13 that contained EcoRI and SacI sites The MuA promoter fragment was subsequently cloned immediately upstream of the ZmFTB fragment using EcoRI and SacI restriction sites in the pGEM-anti-FTB vector to yield the construct MuA-anti-FTB (SEQ ID NO:4) in the pGEM4 vector. A KpnI fragment containing MuA promoter and the corn FTB sequence was PCR amplified from pGEM-MuA-anti-FTB using primers identified by SEQ ID NO:14 and SEQ ID NO:15 and cloned into a binary Ti vector. The construct was transformed into corn via Agrobacterium tumefaciens mediated tissue-culture transformation. A total of 31 independent transgenic events were identified and advanced to produce T2 seeds. Subsequently 7 homozygous transgenic events were isolated by Southern analysis and herbicide selection. The T2 plants are grown to produce T3 homozygous seeds for physiology and field tests. Molecular and genetic analysis is performed.

TABLE 4

MuA-anti-CornFTB Terminator-less
Cassette for plant transformation (SEQ ID NO: 4)
The nucleic acid sequence of pMuA-antisense-cornFT-B-ΔTerm
Bolded sequence is the corn MuA promoter.
Underlined sequence is the corn FT-B antisense sequence.

GAATTCAAATTTTTCGCCAGTTCTAAATATCCGGAAACCTCTTGGGATGCCATTGCCCATCT
ATCTGTAATTTATTGACGAAATAGACGAAAAGGAAGGTGGCTCCTATAAAGCACATCATTGC
GATAACAGAAAGGCCATTGTTGAAGATACCTCTGCTGACATTGGTCCCCAAGTGGAAGCACC
ACCCCATGAGGAGCACCGTGGAGTAAGAAGACGTTCGAGCCACGTCGAAAAAGCAAGTGTGT
TGATGTAGTATCTCCATTGACGTAAGGGATGACGCACAATCCAACTATCCATCGCAAGACCA
TTGCTCTATATAAGAAAGTTAATATCATTTCGAGTGGCCACGCTGAGCTCGGATGGATTGGC
TCCAGCAAATTAGAGTACGGTCCAAGCACATGCTGAGGTAATGGGCACGAACCAGTATCAGT
CATGGCACTGTACTGGCTAACTGCGAGGCCACTGAGGCAGTAGCATGAATGATAGTGATCTC
TGTTCTTTCCAGGCTTATCCCTCAAGCCTCCCTCTAGTACCTGAGAACAAAGTAGGATGTAT
TGTTGCAGGGCAATGTTATGGAAGAGTGGGCCAATTTGGTTGCTCTGTTGTATAAAATCAAA
TCCAAACTTCGCATAGTCCACAGCAGAGGAAGACTTATTCGCGGTGCACCCATATGAACTGG
TGCTGCAGGCATCCTCTCCTGATGGCCTTTTGCAGGAATACGAGGACCTCAATTGCTTATCA
ACAATCGTAATTAACTTTTGTGTGAAAGCAATGGCAGCTCCCTGCCAAAAGGAGTAGCAACC
ATCAACCAATTTATTAGTTCGTCCTTGAAATCCGCATTCCACTCCTTGACGAAAAGCCACCC
AGCCAATCAAACTAGGCAAGTCAACTTTCTCTGCCTCATTAAGCAGGATCAAAGCAGCCAAT
CCACAGAATGTATACCCACCATGTGCTTCAGCATAAGGCTCCCCAGCAATACCACCTTCATA
AGTTTGACATCTTGCTATGTAGTCGCCTACACCTTTTGCCAGTTTAAAATCAAGAATATTCA
CAAGGCTGGCAACCGATATAGCGGTGTAGGAAGCACGGACATCAATTTCGCCACCATCATGC
ATTCTGAAAGCACCTGATACATCTTTCATCTGCAGCATAAAATTGTACAGGTTGCCCCTATT
GATTGATGACAATGCTCTTTCGCTCCCTATTGTCACAAGTGTATTTACAGCAGCATAAGTCG
TAGCTAGGTGAGGCAACTGTCCAGGTCCACCACTATATCCACCATCTTTATCCTGACATCGA
GCTAAGAAGTCTATGATATCATTCTCAAGATCATCATCAAGTGCTTCATCCAGCAAAGCAAG
TGGATGAACCATCCAGTAGCATAGCCAAGGGCGATTGGCATCTAGAACATGAAAGGCTGGTC
CCATATGCCTCAGCCCAGGCGTCAGATACTCGATATGCTGATCACGCCACAGCTCTAGCATG
ATGGATTTCGTGTTGGGCGCGGCCCCGAAGAGGGAGCGGTAGATGTCGCCAACCCTGGCCTC
CACCTTCATCTGCTCCACCTGCGTCACCGTGAGCCTCGGTAGGTCGGGATCC

### EXAMPLE 2

### Transformation

Arabidopsis transgenic plants were produced by the method of dipping flowering plants into an Agrobacterium culture, based on the method of Andrew Bent in, Clough S J and Bent A F, 1998. Floral dipping: a simplified method for Agrobacterium-mediated transformation of Arabidopsis thaliana. Wild type plants were grown under standard conditions with a 16 hour, 8 hour light to dark day cycle, until the plant has both developing flowers and open flowers. The plant was inverted for 2 minutes into a solution of Agrobacterium culture carrying the appropriate gene construct. Plants were then left horizontal in a tray and kept covered for two days to maintain humidity and then righted and bagged to continue growth and seed development. Mature seed was bulk harvested.

T1 plants were germinated and grown on MS plates containing kanamycin (50 μg/ml), and kanamycin resistant T1 seedlings were selected and transferred to soil for further growth. Alternative selective markers can be used as desired by a person of skill in the art. Selection may also be done by a PCR screening mechanism wherein DNA is isolated and PCR analysis done to select those individuals containing a desired nucleic acid sequence or sequences.

Transgenic Brassica napus, Glycine max and Zea maize plants can be produced using Agrobacterium mediated transformation of cotyledon petiole tissue. Seeds are sterilized as follows. Seeds are wetted with 95% ethanol for a short period of time such as 15 seconds. Approximately 30 ml of sterilizing solution I is added (70% Javex, 100 μl Tween20) and left for approximately 15 minutes. Solution I is removed and replaced with 30 ml of solution II (0.25% mecuric chloride, 100 μl Tween20) and incubated for about 10 minutes. Seeds are rinsed with at least 500 ml double distilled sterile water and stored in a sterile dish. Seeds are germinated on plates of ½MS medium, pH 5.8, supplemented with 1% sucrose and 0.7% agar. Fully expanded cotyledons are harvested and placed on Medium I (Murashige minimal organics (MMO), 3% sucrose, 4.5 mg/L benzyl adenine (BA), 0.7% phytoagar, pH5.8). An Agrobacterium culture containing the nucleic acid construct of interest is grown for 2 days in AB Minimal media. The cotyledon explants are dipped such that only the cut portion of the petiole is contacted by the Agrobacterium solution. The explants are then embedded in Medium I and maintained for 5 days at 24° C., with 16, 8 hr light dark cycles. Explants are transferred to Medium II (Medium I, 300 mg/L timentin,) for a further 7 days and then to Medium III (Medium II, 20 mg/L kanamycin). Any root or shoot tissue which has developed at this time is dissected away. Transfer explants to fresh plates of Medium III after 14-21 days. When regenerated shoot tissue develops the regenerated tissue is transferred to Medium IV (MMO, 3% sucrose, 1.0% phytoagar, 300 mg/L timentin, 20 mg/L 20 mg/L kanamycin). Once healthy shoot tissue develops shoot tissue dissected from any callus tissue are dipped in 10× IBA and transferred to Medium V (Murashige and Skooge (MS), 3% sucrose, 0.2 mg/L indole butyric acid (IBA), 0.7% agar, 300 mg/L timentin, 20 mg/L 20 mg/L kanamycin) for rooting. Healthy plantlets are transferred to soil. The above method, with or without modifications, is suitable for the transformation of numerous plant species including Glycine max, Zea maize and cotton.

Transgenic Glycine max, Zea maize and cotton can be produced using Agrobacterium-based methods which are known to one of skill in the art. Alternatively one can use a particle or non-particle biolistic bombardment transformation method. An example of non-particle biolistic transformation is given in U.S. patent application Ser. No. 20010026941.

### EXAMPLE 3

### GUS Assays

Leaf tissue was harvested and incubated with GUS staining solution (50 mM NaPO₄, pH 7.0, 0.1% Triton X-100, 1 mM EDTA, 2 mM DTT, 0.5 mg/mL X-GlcA) and left to incubate overnight at 37° C. The staining solution was replaced with fixation buffer (10% formaldehyde, 50% ethanol) and incubated for 30 minutes at room temperature. The fixation buffer was replaced with 80% ethanol and incubated for 1 hour at room temperature. The 80% ethanol was replaced with 100% ethanol and incubated for 1 hour at room temperature. The tissue was assessed for blue staining, indicating GUS activity.

### EXAMPLE 4

### Transformations (B)

Plasmid pHPR:GUS was transformed into Arabidopsis thaliana, T1 seed collected and transgenic T1 plants selected. Transgenics were selected and advanced on kanamycin and assessed for GUS activity. Transgenic lines were advanced to the T3 generation thereby providing homozygous lines.

Plasmid pBI121:Anti-GUS:ΔTerm was transformed into a T3 generation line of Arabidopsis homozygous for the pHPR:GUS construct. T1 plants were screened for the presence of the pBI121:Anti-GUS:ΔTerm sequence by a pooled PCR method. Three flats (12×24 cells) were seeded. Pooled tissue samples were then collected from each flat by sampling both by row and by column across the flat, thereby generating 36 pools per flat. Control plants, Columbia and pHPR:GUS, were grown in 3″ pots. Individual tissue samples were taken from the control plants. Genomic DNA was extracted and PCR was performed using the primer pair identified by SEQ ID NO:16 and SEQ ID NO:17. Based on pools that indicated a positive PCR result, 29 possible candidates were identified and transplanted into 4″ pots. Individual tissue samples were taken and genomic DNA was extracted for PCR analysis using the primer pair identified by SEQ ID NO:16 and SEQ ID NO:17. A total of 8 plants were found to be positive for the pBI121:Anti-GUS:ΔTerm sequence. The presence of the pHPR:GUS sequence for GUS expression was confirmed by PCR amplification using the primer pair identified by SEQ ID NO:5 and SEQ ID NO:18. Lines 1, 2, 4, 6, 7, 17, 18 and 22 were identified.

The cassette MuA:ZmFT-B:ΔTerm was transformed into corn. Positive transformants were selected by herbicide resistance and the presence of the construct confirmed by PCR. Physiological and molecular analysis was performed

### EXAMPLE 5

### Arabidopsis Transgenics pHPR:GUS+pBI121:Anti-GUS:ΔTerm

Molecular Analysis of pHPR:GUS+pBI121:Anti-GUS:ΔTerm:

The eight T1 lines identified by the screening protocol were advanced to the T2 generation. Molecular analysis was again performed on lines 17, 18 and 22 to ensure the constructs were heritable and the lines were zygosity. Genomic DNA was isolated from leaf tissue and subjected to PCR analysis using the primer pair identified by SEQ ID NO:16 and SEQ ID NO:17 to confirm the presence of the pBI121:Anti-GUS:ΔTerm construct and the primer pair identified by SEQ ID NO:5 and SEQ ID NO:18 to confirm the pHPR:GUS construct.

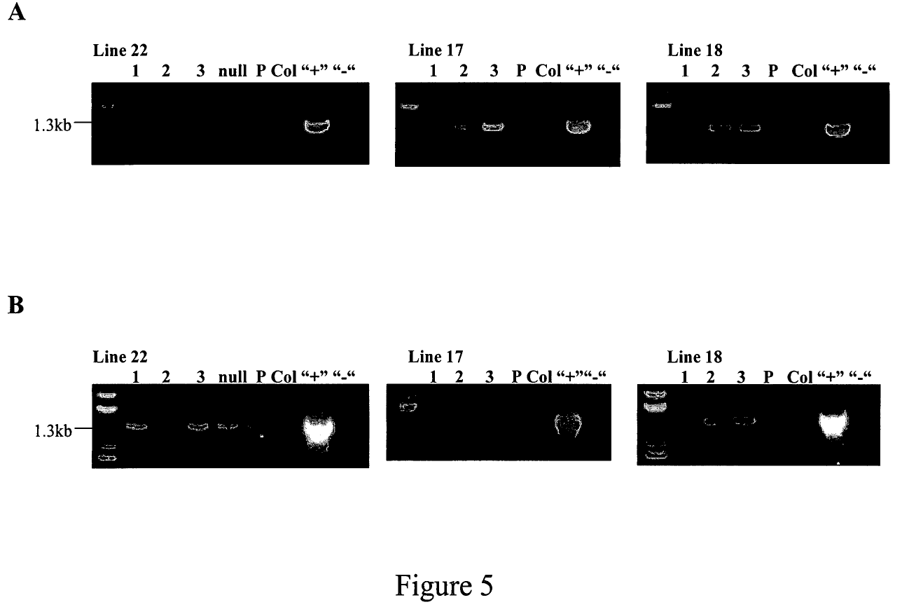

Three lines were selected for detailed characterization in the T2 generation. PCR analysis for construct presence is presented in FIG. 5. Three or four siblings of each line were analyzed and compared to the parental pHPR:GUS control and the Arabidopsis wild-type. Three siblings of lines 17 and 18 and four siblings of line 22 were advanced. PCR analysis using primer pairs SEQ ID NO:5 and SEQ ID NO:18 demonstrated that all lines including the parental controls were transgenic for the pHPR:GUS construct. Columbia controls did not. PCR analysis using the primer pairs SEQ ID NO:16 and SEQ ID NO:17 identified the T2 lines containing the pBI121:Anti-GUS:ΔTerm construct as follows. Lines 17 and 18 three of three siblings carried the antisense construct and three of four siblings of line 22 were positive. The PCR negative line is believed to represent a segregated null.

GUS Assays Results

T1 Plants:

Leaf tissue was harvested from wild type controls, HPR:GUS transgenic plants and HPR:GUS+pBI121:Anti-GUS:ΔTerm transgenic plants. Homozygous HPR:GUS transgenic plants, representing the genetic background line, stained blue throughout the leaf tissue. In contrast, multiple independent transgenic lines of HPR:GUS+pBI121:Anti-GUS:ΔTerm and wild type controls showed a range reduction in HPR gene expression from no observable GUS staining (line 7 and 18) to partial activity or localized activity. For example lines 1 and 22 had activity localized to the leaf vascular tissue, although to a lesser extent than parental controls. Lines 2, 4 and 6 showed diffuse GUS activity however to a significantly reduced level relative to parental control lines. The results indicate that staining, attributable to the HPR:GUS construct was reduced by the pBI121:Anti-GUS:ΔTerm construct.

T2 Plants:

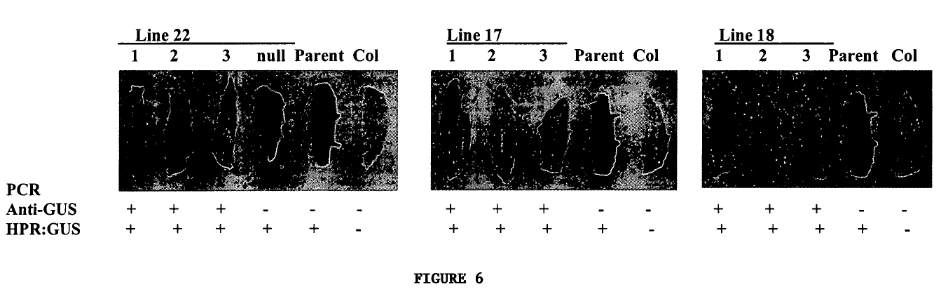

GUS activity analysis of T2 leaf tissue from three sibling plants of the pBI121:Anti-GUS:ΔTerm lines 17 and 18 indicated an absence of GUS activity. Neither was GUS activity detected in the wild-type control plant tissue. However, the GUS activity stain was positive in leaf tissue for the parental control line harboring the pHPR:GUS construct (FIG. 6).

The GUS stain was positive for GUS activity in T2 leaf tissue of one sibling plant of line 22, the segregated null, and the control parent line pHPR:GUS. GUS staining was negative in leaf tissue in line 22, siblings 1, 2, and 3 and also for the wild-type control and the negative experimental control. (FIG. 6).

Hence, introduction of a terminatorless anti-sense-GUS construct efficiently reduces GUS gene expression driven by the HPR promoter. The results demonstrate a terminator region is not a necessary element for anti-sense reduction of gene expression in plants.

### EXAMPLE 6

### Analysis of Corn Transgenics

Physiological data of the lines having at least a copy of the expression cassette identified by SEQ ID NO:4, a terminatorless antisense construct, indicate that relative to the parental control there are identifiable differences in gas exchange properties during vegetative growth, water loss and stomata status, and water transpiration characteristics which are consistent with down regulation of a farnesyl transferase β-subunit. Thereby indicating that the targeted gene has been down-regulated by the pMuA:anti-FT-B:Δterm construct and that the lack of a transcription terminator is not deleterious to the functionality of the antisense method. Characteristics of plants having down-regulated FT-B subunit is described in detail in U.S. application Ser. Nos., 20010044938, 20030061636 and 20040010821.

Plant Growth Conditions

Experiments were conducted in controlled-environment growth cabinet. Plants were grown in 6-L plastic pails filled with “Turface” under a 26/16-° C. day/night temperature regime, 16-h photoperiod, 75% relative humidity, and 600 μmol m⁻² s⁻¹ incident photosynthetic photon flux density (PPFD) at the top of the crop canopy. Four drainage holes were drilled at the base of the side of each pail for drainage. Three seeds per pot were planted, and seedlings were thinned at the 3-leaf stage to one per pail. Pails were watered daily using a nutrient solution as described by Tollenaar (1989, Crop Science 29, 1239-1246).

Gas Exchange

Nine transgenic corn inbred lines and one parental control line were arranged in a randomized complete block design with eight replicates. At the 8^{th} leaf stage water and nutrient solution supply was withheld and pots were covered with aluminum foil to limit evaporation. Plants were supplied with 150 g water on the second and third day following cessation of watering. Leaf gas exchange rate (CER) was measured with a portable, open-flow gas exchange system LI-6400 at 600 μmol m⁻² s⁻¹ PPFD at the leaf surface using the 6400-02 LED light source. The gas exchange rate measured on day 0, prior to stress imposition, was considered as the optimal leaf gas exchange rate under ideal growth conditions. Leaf photosynthesis, stomatal conductance and leaf transpiration during the water stress treatment was calculated using the LI-6400's operating software. Transgenic lines showed a more pronounced inhibition of test parameters. For example, on day 2 of the stress period; leaf photosynthesis of the transgenic line was 62% of optimal compared to 84% of optimal in the parental line; leaf stomatal conductance of the transgenic line was 58% of optimal compared to 94% of optimal in the parental line; and leaf transpiration of the transgenic line was 45% of optimal compared to 83% of optimal in the parental line. Thus the transgenic line demonstrated characteristics consistent with a plant having increased sensitivity to water stress and a greater magnitude of stress responses relative to the parental line. Such responses are expected in a plant having reduced expression of a farnesyl transferase β-subunit.

Water use was determined on a daily basis by weighing of the pots. Total dry biomass was determined at the end of treatment period. Daily water use, defined as the ratio of water lost during stress period over the total dry weight, was calculated. The transgenic line had slightly lower water transpired, greater water in soil and a lower ratio of total water lost relative to final dry weight.

Water Loss and Stomata Status

Water loss was determined on a daily basis by determining the weight of each plant and pot and assuming that the weight loss is due to water use and that water is lost through the stomata. Under water stress conditions the stomata close done thereby reducing the water loss. Nine transgenic inbred corn lines and one parental control line were arranged in a randomized complete block design with eight replicates in each of a water stress group and an unstressed optimal growth conditions group. Water stress was imposed at the 6^{th} leaf stage by cessation of the water/nutrient solution supply and pots were covered with aluminum foil to limit evaporation. Soil water content was maintained in the optimal group by covering pots with aluminum foil to limit evaporation and pots were weighed on a daily basis to estimate water use. Water was supplied to each plant based on the estimated water use. Water loss in the transgenic plants was approximately 83% of the parental water loss on day 2 of the stress period and 65% of parental water loss on day 3 of the stress period. Hence, the reduced water loss in the transgenic lines is indicative of greater degree of stomatal closure as predicted for a down-regulated FT-B line.

Other embodiments are within the following claims.

1. A method of reducing expression of an endogenous gene within a plant cell, the method comprising introducing to said plant cell a gene construct comprising a promoter which is functional within a plant cell operably linked to a nucleic acid sequence, wherein said gene construct lacks a termination region, to obtain a transformed plant cell, wherein transcription of the nucleic acid sequence in said plant cell produces a RNA transcript complementary to an endogenous RNA transcript produced by said endogenous gene thereby reducing the expression of said endogenous gene in said transformed plant cell.

2. The method of claim 1, further comprising growing said transformed plant.

3. The method of claim 1, wherein said endogenous RNA is mRNA.

4. The method of claim 1, wherein said promoter is constitutive promoter, a tissue specific promoter or an inducible promoter

5. The method of claim 1, wherein said nucleic acid is single stranded or double stranded

6. The method of claim 1, wherein said endogenous genes is a metabolic gene, a structural gene a regulatory gene.

7. The method of claim 1, wherein said plant cell is a monocotyledon.

8. The method of claim 1, wherein said plant cell is a dicotyledon

9. The method of claim 1, wherein said plant cell is a gymnosperm.

10. A plant cell wherein expression of an endogenous gene in said plant cell is reduced by the method of claim 1.

11. A plant produced from the cell of claim 10.

12. The plant of claim 10, wherein said plant has reduced expression of said endogenous gene compared to a wild type plant.

13. The plant of claim 10, wherein said plant has an altered stress resistance, altered pathogen resistance, altered herbicide resistance, altered flower color, altered water use, altered transpiration rates, increased fruit production, increased seed production, increase flower production, or increased yield.

14. The plant of claim 11 wherein the plant is a dicotyledonous plant.

15. The plant of claim 11, wherein the plant is a monocotyledonous plant.

16. A seed produced from the plant of claim 11, wherein said seed produces a plant that has reduced expression of said endogenous gene or an altered phenotype compared to a wild-type plant.
